(19) United States
(12) Patent Application Publication
(10) Pub. No.: US 2023/0142317 A1
(43) Pub. Date: May 11, 2023

(54) METHODS OF PREDICTING DISEASE PROGRESSION IN RHEUMATOID ARTHRITIS

(71) Applicant: Laboratory Corporation of America Holdings, Burlington, NC (US)

(72) Inventors: Darl Flake, Salt Lake City, UT (US); Eric Sasso, South San Francisco, CA (US)

(21) Appl. No.: 17/917,887

(22) PCT Filed: Apr. 8, 2021
(86) PCT No.: PCT/US2021/026405

§ 371 Date: Oct. 7, 2022

Related U.S. Application Data
(60) Provisional application No. 63/007,812, filed on Apr. 9, 2020.

(51) Int. Cl. G16H 50/50

(52) U.S. Cl. CPC G16H 50/50

(57) ABSTRACT

Methods for evaluating the risk of radiographic progression in a subject with RA are provided. Also provided are methods of determining treatment efficacy for a subject with RA. Methods of selecting a treatment regimen for a subject with RA are also provided.

### RELATED APPLICATIONS

This application claims priority to U.S. Provisional Application No. 63/007,812, filed Apr. 9, 2020, the entire contents of which are hereby incorporated by reference in their entirety.

### FIELD OF INVENTION

The present invention relates to the fields of inflammatory and autoimmune disease; methods of determining the risk of radiographic progression in RA patients are provided. Also provided are methods of determining treatment regimens that reduce the likelihood of a patient exhibiting one or more adverse effects.

### BACKGROUND

This application is directed to the fields of bioinformatics and inflammatory and autoimmune disease with methods of assessing risk of disease progression in patients with inflammatory diseases including but not limited to rheumatoid arthritis (“RA”). RA is an example of an inflammatory disease and is a chronic, systemic autoimmune disorder. It is one of the most common systemic autoimmune diseases worldwide. The immune system of the RA patient targets the patient's joints, and also causes widespread inflammation that can affect other organs, including the lung, blood vessels and heart. Inflammation of the joints (arthritis) can damage bone with erosions and cause joint space narrowing by damaging cartilage. Joint damage in RA is cumulative and largely irreversible and it may result in permanent disability.

The MBDA score is a validated multi-biomarker disease activity tool that quantifies 12 serum protein biomarkers to assess disease activity in adult patients with RA (Curtis J R, et al., Arthritis Care Res. 64:1794-803 (2012) and US2019/0049443). Derivation of these biomarkers is described in U.S. Pat. No. 9,200,324, herein incorporated by reference in its entirety. Biomarkers can sometimes also be influenced by variables including race, sex, genetics, body mass index, hormones and environmental factors.

Traditional models for treating RA are based on the expectation that controlling disease activity (e.g., inflammation) in an RA subject should slow down or prevent disease progression, in terms of radiographic progression, tissue destruction, cartilage loss and joint erosion. There is evidence, however, that disease activity and disease progression can be uncoupled and may not always function in tandem. Indeed, different cell signaling pathways and mediators are involved in the two processes. See US 2019/0049443, herein incorporated by reference in its entirety and van der Berg et al., Arth Rheum 2005, 52:994-999. The uncoupling of disease progression and disease activity has been described in a number of RA clinical trials and animal studies. See e.g. Lipsky et al., N. Engl. J Med 2003, 343:1594-602, Brown et al., Arth. Rheum. 2006 54:3761-3773 and Pettit et al., Am J. Pathol. 2001, 159:1689-1699. Studies of RA subjects indicate limited association between clinical and radiographic responses. See Zatarain & Strand, Nat. Clin. Pract. Rheum. 2006, 2(11):611-618 (Review). RA subjects have been described who demonstrated radiographic benefits from combination treatment with infliximab and methotrexate (MTX), yet did not demonstrate any clinical improvement, as measured by the 28-joint Disease Activity Score (DAS28) and CRP (C-reactive protein). See Smolen et al., Arth Rheum 2005, 52(4):1020-1030. It has been necessary to track the uncoupling of disease activity and remission and analyze the relationships between disease activity, treatment, and progression of one or more RA-related symptoms.

For patients with eRA, MTX is recommended as first-line treatment and in non-responders both the addition of conventional non-biological disease modifying anti-rheumatic drug therapy (triple DMARD therapy) and of biological (e.g. anti-TNF) therapy are known in the art. The number of drugs available for treating RA is increasing; thus the number of possible drug combinations is increasing as well. Also, the chronological order in which drugs are tried individually or in combination can be varied. Historically these decisions have been made through a trial and error process, but the progression in joint damage is irreversible. See, e.g. Brown et al. Arth Rheum 2008 58(10):2958-2967 and Cohen et al., Ann Rheum Dis. 2007, 66:358-363. The use of disease-modifying anti-rheumatic drug (DMARD) combinations has become accepted for patients who fail to respond to a single DMARD. Studies analyzing treatment with MTX alone and or in combination with other DMARDS suggest that in DMARD-naïve subjects, the balance of efficacy versus toxicity may favor MTX monotherapy, while in DMARD-inadequate responders, the evidence has been inconclusive. Studies support the use of biologics in combination with MTX in subjects with early RA (eRA), in subjects with established RA who have not yet been treated with MTX or in subjects with established RA who failed treatment with MTX monotherapy.

Current clinical management and treatment goals for autoimmune or inflammatory diseases like RA focus on the suppression of disease activity with the goal of improving the subject's functional ability and slowing the progression of joint damage. Clinical assessment of RA disease activity may include measuring the subject's ability to perform daily activities, morning stiffness, pain, the number of tender and swollen joints, and overall global assessments by a medical professional and the patient. Inflammation may be assessed with blood tests that measure the erythrocyte sedimentation rate (ESR) and/or level of CRP. Composite indices comprising multiple variables have been developed as clinical assessment tools to monitor disease activity. Composite indices include, but are not limited to, American College of Rheumatology (ACR) response criteria, Clinical Disease Activity Index (CDAI), DAS, rheumatoid arthritis disease activity index (RA-DAI) and multi-biomarker disease activity (MBDA) score.

Current laboratory tests routinely used to monitor disease activity in RA subjects, such as CRP and ESR, are relatively non-specific, because they can be elevated from conditions other than RA. Conversely, RA subjects may have elevated clinical disease activity despite having ESR and/or CRP that are not elevated and non-RA subjects may display elevated ESR or CRP levels. ESR and CRP are not always accurate for determining response to treatment and they cannot predict future outcomes. As stated above, some subjects in clinical remission, as determined by DAS, continue to demonstrate new joint damage measured with X-rays, called radiographic progression. With biologic drugs, subjects who do not demonstrate clinical benefits may demonstrate radiographic benefits from treatment. See, e.g. Emery et al. J Rheumatol 2009, 36(7):1429-41 and Breedveld et al. Arth Rheum 2006, 54(1):26-37, herein incorporated by reference in their entirety. In contrast, among patients who are responding clinically to treatment, some continue to develop new joint damage, especially with non-biologic drug treatment. The complexity of the response to treatment options and the threat of silent long-term damage to joints provides a significant technical challenge.

Clinical assessments of disease activity contain subjective measurements of inflammatory disease such as RA, such as signs and symptoms, and subject-reported outcomes, which are all difficult to quantify consistently. In clinical trials, the DAS28 is generally used for assessing RA disease activity. The DAS is an index score of disease activity based in part on these subjective parameters. Besides its subjectivity component, another drawback to use of the DAS as a clinical assessment of RA disease activity is its invasiveness. The physical examination required to derive a subject's DAS28 can be painful, because it requires squeezing the subject's joints to assess the amount of tenderness, as measured by the level of discomfort felt by the subject when pressure is applied to the joints. Performing joint counts makes DAS scoring time-consuming. Furthermore, to accurately determine a subject's DAS requires a skilled assessor, to minimize inter- and intra-operator variability.

The present teachings provide methods of assessing risk of radiographic progression, i.e., future joint damage, as a function of inflammatory disease activity and other parameters, utilizing a multivariate model.

The adjusted MBDA score has been shown to be a superior predictor of radiographic progression (RP), both in univariate and multivariate analyses; however, it is also known that negative serologic status, higher BMI and use of biologic therapy are associated with lower rate of RP. Although radiographic progression represents permanent joint damage caused by inadequately controlled rheumatoid arthritis (RA), there is still no standard risk assessment in clinical practice that physicians can use to determine how aggressively to treat a patient's RA to specifically prevent joint damage.

### SUMMARY

Methods of obtaining a radiographic progression (RP) risk score for a subject with rheumatoid arthritis (RA) are provided. A method of obtaining a radiographic progression risk score for a subject with rheumatoid arthritis comprises the steps of obtaining or having obtained a biological sample from the subject, determining the multi-biomarker disease activity score adjusted for age, sex and adiposity (MBDA_{adj}) for the subject, determining at least one of the (a) the serological status of the subject, (b) a BMI surrogate score for the subject and (c) use of targeted therapy, assigning a serological value if the serological status of said subject is determined; assigning a targeted therapy value, if use of targeted therapy is determined; and obtaining an RP risk score from the subject's MBDA_{adj} factor, a BMI surrogate score factor, and one or more values selected from the group consisting of the serological value of the sample and a targeted therapy value using an interpretation function. In aspects of the methods, a high RP risk score indicates an increased risk of ΔTSS >3 in a year. In aspects of the methods, a high RP risk score indicates an increased risk of ΔTSS >5 in a year.

In an embodiment, the application provides methods for determining if a patient having RA is at increased risk of exhibiting an RP-related effect. Methods for determining if a patient having RP is at increased risk of exhibiting an RP-related effect comprise the steps of (i) obtaining or having obtained a biological sample from the subject; (ii) determining the multi-biomarker disease activity score adjusted for age, sex and adiposity (MBDA_{adj}) for the subject, (iii) determining at least one of serological status of the subject, a BMI surrogate score for the subject, and use of a targeted therapy; (iv) assigning a serological value if the serological status of said subject is determined; (v) assigning a targeted therapy value, if use of targeted therapy is determined; and (vi) obtaining an RP risk score from the subject's MBDA_{adj} factor, a BMI surrogate score factor and one or more values selected from the group consisting of the serological value of the sample and a targeted therapy value using an interpretation function, wherein a high RP risk score indicates the patient is at increased risk of exhibiting an RP-related effect. In aspects of the method, the RP-related effect is selected from the group comprising tissue destruction, cartilage loss, synovial fluid build-up, inflammation and joint erosion.

Methods for treating a patient with RA with an appropriate therapy are provided. A method for treating a patient with RA with an appropriate RA therapy comprises determining if the patient has a high RP risk score by obtaining or having obtained a biological sample from the patient, determining the MBDA_{adj} for the patient, determining the serological status of the patient and assigning a serological value to said subject, determining a BMI surrogate score factor, determining if there is use of a targeted therapy and assigning a targeted therapy value; obtaining an RP risk score from the MBDA_{adj}, the BMI surrogate score factor and one or more values selected from the group consisting of the serological value of the sample and a targeted therapy value, using an interpretation function and if the patient has a high RP risk score then administering a different RA therapy to the patient. In an aspect, a high RP risk score indicates an increased risk of ΔTSS >3. In an aspect, a high RP risk score indicates an increased risk of ΔTSS >5. In various aspects, a different RA therapy is selected from the group comprising administering an additional compound, changing a compound, changing the dosing regimen of one or more compounds, or both changing compounds and changing the dosing regimen of one or more compounds. In certain aspects, the risk of an adverse effect for a patient who does not have a high risk of RP is reduced when said RA therapy is reduced and wherein said adverse effect is selected from the group comprising increased risk of infection and hepatoxicity. In some aspects, the patient is at increased risk of exhibiting an RP-related effect. In aspects the risk of exhibiting an RP-related effect is lower when the RA therapy is administered to the patient than it would be if the patient does not receive RA therapy. In various aspects, the RP-related effect is selected from the group comprising tissue destruction, cartilage loss, synovial fluid build-up, inflammation and joint erosion.

Methods of treating a subject with RA are provided. The methods comprise determining if the patient is at an increased risk of exhibiting an RP-related effect comprising obtaining or having obtained a biological sample from the subject; determining the multi-biomarker disease activity score adjusted for age, sex and adiposity (MBDA_{adj}) for the subject; determining at least one of (a) serological status of said subject, (b) a BMI surrogate score for the subject and (c) use of targeted therapy; assigning a serological value, if the serological status of the subject is determined; assigning a targeted therapy value if a use of targeted therapy is determined; and obtaining an RP risk score from the subject's MBDA_{adj} factor, a BMI surrogate score factor and one or more values selected from the group consisting of the serological value of the sample and a targeted therapy value, using an interpretation function; and if the subject has an increased risk of exhibiting an RP-related effect, then administering a different RA therapy.

Methods of monitoring treatment efficacy in a subject with RA are provided. The methods comprise the steps of obtaining or having obtained a first biological sample from a patient at a first time point, determining the multi-biomarker disease activity score adjusted for age, sex and adiposity (MBDA_{adj}) for the patient, determining at least one of a serological status of the patient, a BMI surrogate score for the patient and use of targeted therapy; assigning a serological value if the serological status of the subject is determined; assigning a targeted therapy value if use of targeted therapy is determined; obtaining a first RP risk score from the subject's MBDA_{adj} factor, a BMI surrogate score factor, and one or more values selected from the group consisting of the serological value of the sample and a targeted therapy value, using an interpretation function; administering one or more treatment regimens; obtaining or having obtained a second biological sample from the patient at a second time point; determining the multi-biomarker disease activity score adjusted for age, sex and adiposity (MBDA_{adj}) for the patient, determining at least one of a serological status of the patient, a BMI surrogate score for the patient, and use of targeted therapy; assigning a serological value if the serological status of the subject is determined; assigning a targeted therapy value if use of targeted therapy is determined; obtaining a second RP risk score from the subject's MBDA_{adj} factor, a BMI surrogate score factor and one or more values selected from the group consisting of the serological value of the sample and a targeted therapy value, using an interpretation function, and comparing the first and second RP risk scores to determine treatment efficacy. In various aspects, the change between the first RP risk score and the second RP risk score indicates treatment efficacy. By “treatment efficacy” is intended the ability of a treatment to provide a beneficial effect. A treatment may provide no beneficial effect, that is it may have zero or limited beneficial effect. In such cases the treatment would have a low treatment efficacy. A treatment may have a clinically meaningful beneficial effect or a treatment may have a positive effect on the rate of change. In such cases the treatment would have a high treatment efficacy.

In aspects of any of the methods, the interpretation function is A+(MBDA_{adj} factor)+(serological value)−(BMI surrogate factor)−(targeted therapy value), wherein A is a value selected from the range of 0.7 to 1.2.

Kits for determining an RP risk score for a subject with RA are provided. Kits comprise a sample collection component, a MBDA_{adj} component and a serological status component.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 provides a graph summarizing RP Risk Score with the validation set.

Probability of radiographic progression (RP) at one year at the indicated modified total Sharp score (ΔTSS >3 and ΔTSS >5) are shown. The percentage of patients with each RP Risk score is indicated. As the RP risk score increases, the probability of radiographic progression at one year increases also.

FIG. 2 provides a graph summarizing RP score at ΔTSS >3. The chart provides RP score at ΔTSS >5.

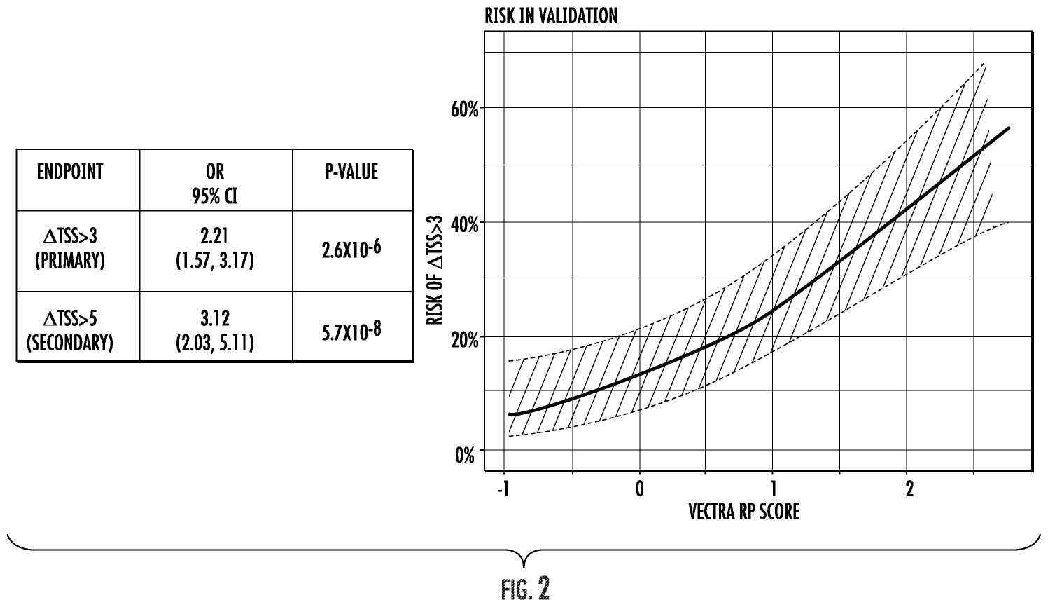

FIG. 3 provides a graph summarizing RP Risk examples for ΔTSS >3 at differing BMI's and with and without targeted therapy.

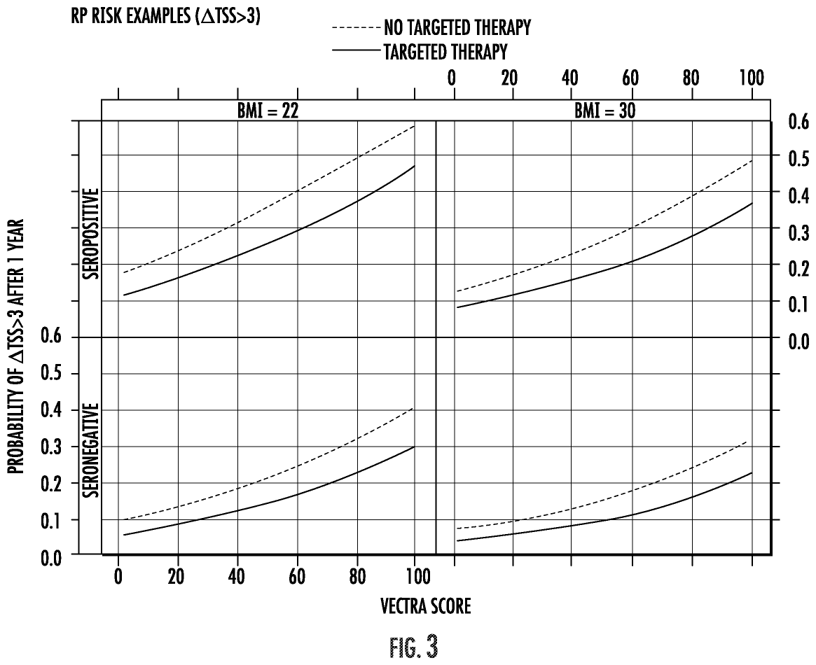

### DETAILED DESCRIPTION

It is commercially desirable to incorporate predictors such as adjusted MBDA score, BMI and serological status to the Vectra Risk Report to predict radiographic progression. The present disclosure provides methods for obtaining a radiographic progression risk score, for determining if a patient having RA is at increased risk of exhibiting an RA-related effect, for treating a patient with an RA therapy, wherein the patient has RA, and for treating a patient with RA, said method comprising determining if a patient having RA is at increased risk of exhibiting an RA-related effect. The multi-biomarker disease activity (MBDA) score, adjusted for age, sex and adiposity (MBDA_{adj}), has been shown to be better than several conventional disease activity measures for predicting risk for radiographic progression (RP) in patients with RA (Curtis, et al. Rheumatology [Oxford]. 2018; 58:874). Serologic status and other non-disease activity measures are also predictive of RP risk. A multivariate tool for obtaining a Radiographic Progression (RP) risk score combining MBDA_{adj}, a BMI surrogate score, a serological value and a targeted therapy value can be used to predict the likelihood a patient will exhibit an inflammatory disease related effect such as but not limited to radiographic progression.

The present methods provide for more accurate determination of a patient or subject's risk of experiencing a rheumatoid arthritis related (RA-related) effect such as, but not limited to, radiographic progression, tissue destruction, cartilage loss, joint erosion and joint deterioration. The development of a multiple biomarker disease activity score (MBDA) for assessing rheumatoid arthritis improved patients and medical practitioners' ability to track and monitor disease progression and optimize treatment plans. However, other factors also impact the risk of radiographic progression. A radiographic progression score based on multivariate inputs allows improved determination of a patient or subject's risk for radiographic progression.

The words “a”, “an” and “the” include plural referents unless the context clearly dictates otherwise.

“Accuracy” refers to the degree that a measured or calculated value conforms to its actual value. “Accuracy” in clinical testing relates to the proportion of actual outcomes (true positives or true negative, wherein a subject is correctly classified as having a risk for a disease related symptom or effect versus incorrectly classified outcomes (false positives or false negatives, wherein a subject is incorrectly classified as at high risk for radiographic progression or as at low to moderate risk for radiographic progression. Other and/or equivalent terms for “accuracy” can include, “specificity”, “positive predictive value (PPV)”, “negative predictive value (NPV)”, “likelihood” and “odds ratio”. “Analytical accuracy” in the context of the present teachings, refers to the repeatability and predictability of the measurement process. Analytical accuracy can be summarized in such measurements as coefficients of variation (CV), tests of concordance, and calibration of the same samples or controls at different times or with different assessors, users, equipment and/or reagents.

The term “administering” as used herein refers to the introduction of a composition into a subject by a method or route that results in at least partial localization of the composition at a desired site such that a desired effect is produced. Routes of administration include both local and systemic administration. Generally local administration results in more of the composition being deliver to a specific location as compared to the entire body of the subject, whereas systemic administration results in delivery to essentially the entire body of the subject.

The term “algorithm” encompasses any formula, model, mathematical equation, algorithmic, analytical or programmed process, or statistical technique or classification analysis that takes one or more inputs or parameters, whether continuous or categorical, and calculates an output value, index, index value or score. Examples of algorithms include but are not limited to ratios, sums, regression operators such as exponents or coefficients, biomarker value transformations and normalizations (including, without limitation, normalization schemes that are based on clinical parameters such as age, gender, ethnicity, etc.), rules and guidelines, statistical classification models, and neural networks trained on populations. Also of use in the context of biomarkers are linear and non-linear equations and statistical classification analyses to determine the relationship between (a) levels of biomarkers detected in a subject sample and (b) the level of the respective subject's risk for exhibiting an inflammatory disease related effect.

The term “analyte” in the context of the present teachings can mean any substance to be measured, and may encompass biomarkers, markers, nucleic acids, electrolytes, metabolites, proteins, sugars, carbohydrates, fats, lipids, cytokines, chemokines, growth factors, peptides, oligonucleotides, mutations, variants, polymorphisms, modifications, fragments, subunits, degradation products and other elements.

To “analyze” includes determining a value or set of values associated with a sample by measurement of analyte levels in the sample. “Analyze” may further comprise comparing a level against a constituent level in a sample or set of samples from the same subject or other subject(s). The biomarkers of the present teachings can be analyzed by any of the various convention methods known in the art. Such methods include, but are not limited to, measuring serum protein or sugar or metabolite or other analyte level, measuring enzymatic activity, and measuring gene expression.

“Autoimmune disease” encompasses any disease as defined herein resulting from an immune response against substances or tissues normally present in the body. Examples of suspected or known autoimmune diseases include rheumatoid arthritis, early rheumatoid arthritis, axial spondyloarthritis, juvenile idiopathic arthritis, seronegative spondyloarthropathies, ankylosing spondylitis, psoriatic arthritis, antiphospholipid antibody syndrome, autoimmune hepatitis, Behcet's disease, bullous pemphigoid, coeliac disease, Crohn's disease, dermatomyositis, Goodpasture's syndrome, Grave's disease, Hashimoto's disease, idiopathic thrombocytopenic purpura, IgA nephropathy, Kawasaki disease, systemic lupus erythematosus, mixed connective tissue disease, multiple sclerosis, myasthenia gravis, polymyositis, primary biliary cirrhosis, psoriasis, scleroderma, Sjogren's syndrome, ulcerative colitis, vasculitis, Wegener's granulomatosis, temporal arteritis, Takayasu's arteritis, Henoch-Schonlein purpura, leucocytoclastic vasculitis, polyarteritis nodosa, Churg-Strauss Syndrome, and mixed cryoglobulinemic vasculitis.

A “biologic” or “biotherapy” or “biopharmaceutical” is a pharmaceutical therapy product manufactured or extracted from a biological substance. A biologic can include vaccines, blood or blood components, allergenics, somatic cells, gene therapies, tissues, recombinant proteins, and living cells; and can be composed of sugars, proteins, nucleic acids, living cells or tissues or combinations thereof. Examples of biologic drugs can include vaccines, blood or blood components, allergenics, somatic cells, gene therapies, tissues, recombinant proteins, and living cells; and can be composed of sugars, proteins, nucleic acids, living cells or tissues, or combinations thereof. Examples of biological drugs can include but are not limited to biological agents that target the tumor necrosis factor (TNF)-alpha molecules, such as the TNF inhibitors infliximab, adalimumab, etanercept, certolizumab and golimumab. Other classes of biologic drugs include IL-1 inhibitors such as anakinra, T-cell modulators such as abatacept, B-cell modulators such as rituximab, and IL-6 inhibitors such as tocilizumab and sarilumab.

“Biomarker”, “biomarkers”, “marker” or “markers” in the context of the present teachings encompasses, without limitation, cytokines, chemokines, growth factors, proteins, peptides, nucleic acids, oligonucleotides, and metabolites, together with their related metabolites, mutations, isoforms, variants, polymorphisms, modifications, fragments, subunits, degradation products, elements, and other analytes or sample-derived measures. Biomarkers can also include mutated proteins, mutated nucleic acids, variations in copy numbers and/or transcript variants. Biomarkers also encompass non-blood borne factors and non-analyte physiological markers of health status and/or other factors or markers not measured from samples (e.g. biological samples such as bodily fluids), such as clinical parameters and traditional factors for clinical assessments. Biomarkers can also include any indices that are calculated and/or created mathematically. Biomarkers can also include combinations of any one or more of the foregoing measurements, including temporal trends and differences. Where the biomarkers of certain embodiments of the present teachings are proteins, the gene symbols and names used herein are to be understood to refer to the protein products of these genes, and the protein products of these genes are intended to include any protein isoforms of these genes, whether or not such isoform sequences are specifically described herein. Biomarkers can include but are not limited to the biomarkers described in WO2019055609.

A “clinical assessment”, “clinical datapoint” or “clinical endpoint” in the context of the present teachings can refer to a measure of disease activity or severity. A clinical assessment can include a score, a value, or a set of values that can be obtained from evaluation of a sample (or population of samples) from a subject or subjects under determined conditions. A clinical assessment can also be a questionnaire completed by a subject. A clinical assessment can also be predicted by biomarkers and/or other parameters. One of skill in the art will recognize that the clinical assessment for RA, as an example, can comprise without limitation one or more of the following DAS, DAS28, DAS28-ESR, DAS28-CRP, health assessment questionnaire (HAQ), modified HAQ (mHAQ), multi-dimensional HAQ (MDHAQ), visual analog scale (VAS), physician global assessment VAS, patient global assessment VAS, pain VAS, fatigue VAS, overall VAS, sleep VAS, simplified disease activity index (SDAI), clinical disease activity index (CDAI), routine assessment of patient index data (RAPID), RAPID3, RAPID4, RAPID5, RA-DAI, American College of Rheumatology (ACR) responses, such as ACR20, ACR50, ACR70, ACRn, SF-36 (a well-validated measure of general health status), RA MRI score (RAMRIS; or an RA MRI scoring system), total Sharp score (TSS), van der Heijde-modified TSS, van der Heijde-modified Sharp score (or Sharp-van der Heijde [SHS]), Larsen score, TJC, swollen joint count (SJC), CRP titer (or level), erythrocyte sedimentation rate (ESR), MBDA score and the adjusted MBDA score (MBDA_{adj}).

The term “clinical variable” or “clinical parameters” in the context of the present teachings encompasses all measures of the health status of a subject. A clinical parameter can be used to derive a clinical assessment of the subject's disease activity. Clinical parameters can include, without limitation: therapeutic regimen (including but not limited to DMARDs, whether conventional or biologic, steroids, etc.), TJC, SJC, morning stiffness, arthritis of three or more joint areas, arthritis of hand joints, symmetric arthritis, rheumatoid nodules, radiographic changes and other imaging, gender/sex, smoking status, age, race/ethnicity, disease duration, diastolic and systolic blood pressure, resting heart rate, height, weight, adiposity, body-mass index, serum leptin, family history, CCP status (ie. whether subject is positive or negative for anti-CCP antibody), CCP titer, RF status, RF titer, ESR, CRP, menopausal status, and whether a smoker/non-smoker.

“Clinical assessment’ and “clinical parameter” are not mutually exclusive terms. There may be overlap in members of the two categories. For example, CRP concentration can be used as a clinical assessment of disease activity; or, it can be used as a measure of the health status of a subject and thus serve as a clinical parameter.

“DAS” refers to the Disease Activity Score, a measure of the activity of RA in a subject, well-known to those of skill in the art. See van der Heijde et al, Ann. Rheum. Dis 1990, 49(11):916-920. The DAS28 involves the evaluation of 28 specific joints. DAS may refer to calculations based on 66/68 or 44 joint counts and is used less often than the DAS28. DAS28 is intended to include but is not limited to, DAS28, DAS28-ESR and DAS28-CRP. A DAS28 can be calculated for an RA subject by any means known in the art. The number of swollen joints, or swollen joint count out of a total of 28 (SJC28), and tender joints, or tender joint count out of a total of 28 (TJC28) in each subject, is assessed. In some DAS28 calculations the subject's general health (GH) is also a factor and may be measured on a 100 mm Visual Analogue Scale (VAS). GH may also be referred to as PG or PGA. A “patient global health assessment VAS” is GH measured on a Visual Analogue Scale.

The DAS28-ESR or DAS28ESR is a DAS28 assessment wherein the ESR for each subject is measured in mm/hour. The DAS28-ESR can be calculated by any formula known in the art. DAS28-CRP or DAS28CRP is a DAS28 assessment calculated using CRP in place of ESR. CRP is produced in the liver. Normally CRP circulating in a subject's blood serum is at low levels. CRP levels often increase during episodes of acute inflammation or infection, so that a high or increasing amount of CRP in blood serum can be associated with acute infection or inflammation. A blood serum level of CRP greater than 1 mg/dL is usually considered high. Inflammation and infections may result in CRP levels greater than 10 mg/dL. The level of CRP in subject sera can be quantified using any method known in the art including but not limited to, ELISA assays. Elevated CRP levels are associated with increased risk for radiographic progression in RA and may be incorporated into a multivariate analysis RP risk score. The DAS28-CRP can be calculated by any formula known in the art.

A “difference” as used herein refers to an increase or decrease in the measurable expression of a biomarker or panel of biomarkers as compared to the measurable expression of the same biomarker or panel of biomarkers in a second sample.

The term “disease” in the context of the present teachings encompasses any disorder, condition, sickness, ailment, etc. that manifests in, e.g., a disordered or incorrectly functioning organ, part, structure or system of the body, and results from e.g. genetic or developmental errors, inflammatory or autoimmune processes, infection, poisons, nutritional deficiency or imbalance, toxicity or unfavorable environmental factors.

A DMARD can be conventional synthetic or biologic or targeted synthetic. Examples of DMARDS that are generally considered conventional synthetic include, but are not limited to, MTX, azathioprine (AZA), bucillamine (BUC), chloroquine (CQ), ciclosporin (CSA, or cyclosporine, or cyclosporin), doxycycline (DOXY), hydroxychloroquine (HCQ), intramuscular gold (IM gold), leflunomide (LEF), levofloxacin (LEV) an sulfasalazine (SSZ). Examples of other conventional synthetic DMARDs include, but are not limited to, folinic acid, D-pencillamine, gold auranofin, gold aurothioglucose, gold thiomalate, cyclophosphamide, and chlorambucil. Examples of biological DMARDs (or biologic drugs) include but are not limited to biological agents that target the tumor necrosis factor (TNF)-alpha molecules such as infliximab, adalimumab, etanercept and golimumab. Other classes of biologic DMARDs include IL-1 inhibitors such as, but not limited, anakinra, T-cell modulators such as abatacept, B-cell modulators such as rituximab, and IL-6 pathway inhibitors such as tocilizumab and sarilumab. Targeted synthetic DMARDs include but are not limited to Janus kinase (JAK) inhibitors, such as tofacitinib, baricitinib and others.

The term “flare” as used herein is an increase in the level of symptoms and clinical manifestations including, but not limited to, an increase in SJC, increase in TJC, increase in serologic markers of inflammation (e.g. CRP and ESR), decrease in subject function (e.g. ability to perform basic daily activities), increase in morning stiffness, and increases in pain that commonly lead to therapeutic intervention and potentially to treatment intensification. Flare onset may be sudden onset or gradual.

The term “infection” as used herein refers to an infection that leads to fever, disease or requires medical intervention including, but not limited to, antibiotic use. “Infection” is intended to encompass infection and “serious infection”. The term “serious infection” as used herein refers to an infection that leads to death, hospitalization or requires intravenous antibiotics. Serious infections include, but are not limited to bacterial infections, Mycobacterium tuberculosis and other mycobacterial infections, invasive pneumococcal disease, pneumonia, septicemia and bacteremia, invasive bacterial infection after chemotherapy, neonatal septicemia, meningitis, encephalitis, bone and joint sepsis, severe cutaneous infections cellulitis, urosepsis, bowel and other GI tract infections, severe viral infections and opportunistic infections, especially fungal infections.

“Inflammatory disease” includes, but is not limited to, any disease resulting from the effect a biological response to a stimulus may have on vascularized tissues, including but not limited to such stimuli as pathogen, damaged cells, irritants, antigens and in the case of autoimmune disease, substances and tissues normally present in the body. Non-limiting examples of inflammatory disease include but are not limited to rheumatoid arthritis (RA), eRA, ankylosing spondylitis, psoriatic arthritis, atherosclerosis, asthma, autoimmune disease, chronic inflammation, chronic prostatitis, glomerulonephritis, hypersensitivities, inflammatory bowel diseases, pelvic inflammatory disease, reperfusion injury, transplant rejection and vasculitis.

“Measuring” or “measurement” refers to determining the presence, absence, quantity, amount, or effective amount of a substance in a clinical or subject-derived sample, including the concentration levels of such substances or evaluating the values or categorization of a subject's clinical parameters.

By a “multi-biomarker disease activity score”, “multi-biomarker disease activity index score”, “MBDA score” or simply “MBDA” is intended a score that provides a quantitative measure of inflammatory disease activity or the state of inflammatory disease in a subject. A set of data from particularly selected biomarkers, such as from the set of biomarkers disclosed in WO2019055609 is input into an interpretation function according to the present teachings to derive the MBDA score. The interpretation function, in some embodiments, can be created from predictive or multivariate modeling based on statistical algorithms. Input to the interpretation function can comprise the results of testing two or more biomarkers alone or in combination with clinical parameters and/or clinical assessments, also described herein. In some embodiments the MBDA score is a quantitative measure of autoimmune disease activity. In some embodiments the MBDA score is a quantitative measure of RA disease activity. MBDA as used herein can refer to a Vectra DA score, also known as a Vectra score.

In some embodiments, the interpretation function is based on a predictive model. Established statistical algorithms and methods well-known in the art, useful as models or useful in designing predictive models, can include but are not limited to: analysis of variants (ANOVA); Bayesian networks; boosting and Ada-boosting; bootstrap aggregating (or bagging) algorithms; decision trees classification techniques, such as Classification and Regression Trees (CART), boosted CART, Random Forest (RF), Recursive Partitioning Trees (RPART), and others; Curds and Whey (CW); Curds and Whey-Lasso; dimension reduction methods, such as principal component analysis (PCA) and factor rotation or factor analysis; discriminant analysis, including Linear Discriminant Analysis (LDA), Eigengene Linear Discriminant Analysis (ELDA), and quadratic discriminant analysis; Discriminant Function Analysis (DFA); factor rotation or factor analysis; genetic algorithms; Hidden Markov Models; kernel based machine algorithms such as kernel density estimation, kernel partial least squares algorithms, kernel matching pursuit algorithms, kernel Fisher's discriminate analysis algorithms, and kernel principal components analysis algorithms; linear regression and generalized linear models, including or utilizing Forward Linear Stepwise Regression, Lasso (or LASSO) shrinkage and selection method, and Elastic Net regularization and selection method; glmnet (Lasso and Elastic Net-regularized generalized linear model); Logistic Regression (Log Reg); meta-learner algorithms; nearest neighbor methods for classification or regression, e.g. Kth-nearest neighbor (KNN); non-linear regression or classification algorithms; neural networks; partial least square; rules based classifiers; shrunken centroids (SC); sliced inverse regression; Standard for the Exchange of Product model data, Application Interpreted Constructs (StepAIC); super principal component (SPC) regression; and, Support Vector Machines (SVM) and Recursive Support Vector Machines (RSVM), among others. Additionally, clustering algorithms as are known in the art can be useful in determining subject sub-groups.

Logistic Regression is the traditional predictive modeling method of choice for dichotomous response variables; e.g., treatment 1 versus treatment 2. It can be used to model both linear and non-linear aspects of the data variables and provides easily interpretable odds ratios.

Discriminant Function Analysis (DFA) uses a set of analytes as variables (roots) to discriminate between two or more naturally occurring groups. DFA is used to test analytes that are significantly different between groups. A forward step-wise DFA can be used to select a set of analytes that maximally discriminate among the groups studied. Specifically, at each step all variables can be reviewed to determine which will maximally discriminate among groups. This information is then included in a discriminative function, denoted a root, which is an equation consisting of linear combinations of analyte concentrations for the prediction of group membership. The discriminatory potential of the final equation can be observed as a line plot of the root values obtained for each group. This approach identifies groups of analytes whose changes in concentration levels can be used to delineate profiles, diagnose and assess therapeutic efficacy. The DFA model can also create an arbitrary score by which new subjects can be classified as either “healthy” or “diseased.” To facilitate the use of this score for the medical community the score can be rescaled so a value of 0 indicates a healthy individual and scores greater than 0 indicate increasing risk.

Classification and regression trees (CART) perform logical splits (if/then) of data to create a decision tree. All observations that fall in a given node are classified according to the most common outcome in that node. CART results are easily interpretable—one follows a series of if/then tree branches until a classification results.

Support vector machines (SVM) classify objects into two or more classes. Examples of classes include sets of treatment alternatives, sets of diagnostic alternatives, or sets of prognostic alternatives. Each object is assigned to a class based on its similarity to (or distance from) objects in the training data set in which the correct class assignment of each object is known. The measure of similarity of a new object to the known objects is determined using support vectors, which define a region in a potentially high dimensional space (>R6).

The process of bootstrap aggregating, or “bagging,” is computationally simple. In the first step, a given dataset is randomly resampled a specified number of times (e.g., thousands), effectively providing that number of new datasets, which are referred to as “bootstrapped resamples” of data, each of which can then be used to build a model. Then, in the example of classification models, the class of every new observation is predicted by the number of classification models created in the first step. The final class decision is based upon a “majority vote” of the classification models; i.e., a final classification call is determined by counting the number of times a new observation is classified into a given group, and taking the majority classification (33%+ for a three-class system). In the example of logistical regression models, if a logistical regression is bagged 1000 times, there will be 1000 logistical models, and each will provide the probability of a sample belonging to class 1 or 2.

Curds and Whey (CW) using ordinary least squares (OLS) is another predictive modeling method. See L. Breiman and J H Friedman, J. Royal. Stat. Soc. B 1997, 59(1):3-54. This method takes advantage of the correlations between response variables to improve predictive accuracy, compared with the usual procedure of performing an individual regression of each response variable on the common set of predictor variables X. In CW, Y=XB*S, where Y=(yₖⱼ) with k for the k^{th} patient and j for j^{th} response (j=1 for TJC, j=2 for SJC, etc.), B is obtained using OLS, and S is the shrinkage matrix computed from the canonical coordinate system. Another method is Curds and Whey and Lasso in combination (CW-Lasso). Instead of using OLS to obtain B, as in CW, here Lasso is used, and parameters are adjusted accordingly for the Lasso approach.

Many of these techniques are useful either combined with a biomarker selection technique (such as, for example, forward selection, backwards selection, or stepwise selection), or for complete enumeration of all potential panels of a given size, or genetic algorithms, or they can themselves include biomarker selection methodologies in their own techniques. These techniques can be coupled with information criteria, such as Akaike's Information Criterion (AIC), Bayes Information Criterion (BIC), or cross-validation, to quantify the tradeoff between the inclusion of additional biomarkers and model improvement, and to minimize overfit. The resulting predictive models can be validated in other studies, or cross-validated in the study they were originally trained in, using such techniques as, for example, Leave-One-Out (LOO) and 10-Fold cross-validation (10-Fold CV).

The MBDA_{adj} is an MBDA score adjusted for age, sex, and adiposity. In some instances, leptin is a surrogate for adiposity. MBDA_{adj}, has been shown to be better than several conventional disease activity measures for predicting risk for radiographic progression (RP) in patients with rheumatoid arthritis (RA) (Curtis, et al. Rheumatology [Oxford]. 2018; 58:874).

The terms “normal”, “control”, and “healthy,” refer generally to a subject or individual (or group thereof) who does not have, is not/has not been diagnosed with, or is asymptomatic for a particular disease or disorder. The terms can also refer to a sample obtained from such subject or individual. The disease or disorder under analysis or comparison is a determinative of whether the subject is a “control” in that situation.

By “prognosis” is intended a prediction as to the likely outcome of a disease. Prognostic estimates are useful in, among other things, determining an appropriate therapeutic regimen for a subject.

The term “remission” refers to the state of absence of disease activity in patients known to have a chronic illness that usually cannot be cured. Remission in RA may be achieved by meeting a formal definition, such as but not limited to DAS28<2.6 or the ACR remission criteria, or by meeting less formal criteria, such as but not limited to clinician impression that a patient is in remission. The term “sustained clinical remission” or “SC-REM” as used herein refers to state of clinical remission sustained as evaluated based on clinical assessments, for example, DAS28 for at least 6 months. The term “functional remission” as used herein refers to a state of remission as evaluated using functional assessment measures such as but not limited to HAQ. Sustained remission can be used interchangeably with maintained remission.

A “sample” in the context of the present teachings refers to any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between or external to the tissues that produce them, including synovial fluid, gingival crevicular fluid, bone marrow, cerebrospinal fluid (CSF), saliva, mucous, sputum, semen, sweat, urine or any other bodily fluids. “Blood sample” can refer to whole blood or any fraction thereof, including but not limited to blood cells, red blood cells, white blood cells, platelets, serum and plasma. Samples can be obtained from a subject by any means known in the art including, but not limited to, venipuncture, excretion, ejaculation, massage, biopsy, needle aspirate, lavage, scraping, surgical incision or intervention or other means known in the art.

By “score” is intended a value or set of values selected to provide a quantitative measure of a variable or characteristic of a subject's condition, and/or to discriminate, differentiate or otherwise characterize a subject's condition. The value(s) comprising the score can be based on, for example, quantitative date resulting in a measured amount of one or more sample constituents obtained from the subject, or from clinical parameters, or from clinical assignments or any combination thereof. In certain embodiments a score can be derived from a single constituent, parameter, assessment, or score, while in other embodiments the score is derived from multiple constituents, parameters, assessments or scores. The score can be based upon or derived from an interpretation function; e.g. an interpretation function derived from a particular predictive model using and of various statistical algorithms known in the art. A score may comprise a factor, wherein said factor is a value obtained from at least one interpretive function and at least one measurement, assessment or categorical input based on at least one clinical parameter. By a “change in score” is intended to encompass an absolute change in score such as, but not limited to, from a first time point to a second time point, a percentage change in the score, and a change in the score per unit time (for example, the rate of score change). By “radiographic progression risk score” or “RP risk score” is intended a score that uses quantitative data to provide an indicator for the risk of radiographic progression over the year following the determination of the score. The risk score provides an estimate of the probability the subject will experience measurable radiographic progression within one year. A set of data from particularly selected biomarkers and clinical values is input into an interpretive function to obtain the RP risk score. The higher the RP risk score, the higher the likelihood or probability the subject will have or will continue to have radiographic progression. A high RP risk score may be an RP risk score in the top 0.5%, top 1%, top 2%, top 3%, top 4%, top 5%, top 6%, top 7%, top 8%, top 9%, top 10%, top 12%, top 14%, top 16%, top 18%, top 20%, top 25%, top 30%, top 40%, top 45%, top 50%, or top 55% of the RP risk score range. A low RP risk score may be an RP risk score in the bottom 0.5%, bottom 1%, bottom 2%, bottom 3%, bottom 4%, bottom 5%, bottom 6%, bottom 7%, bottom 8%, bottom 9%, bottom 10%, bottom 12%, bottom 14%, bottom 16%, bottom 18%, bottom 20%, bottom 25%, bottom 30%, bottom 40%, bottom 45%, bottom 50%, or bottom 55% of the RP risk score range.

In an embodiment the RP risk score may have a score range from −1 to 3. A high RP risk score may be an RP risk score greater than about 0.29, 0.38, 0.48, 0.58, 0.68, 0.78, 0.88, 0.98, 1.08, 1.18, and 1.2 when the RP risk score range is −1 to 3. A high RP risk score may correspond to at least a 6% risk for RP >5 Sharp units or at least a 16% risk for RP >3 Sharp units. A high RP risk score may correspond to at least a 10% risk for RP >5 Sharp units or at least a 22% risk for RP >3 Sharp units. A high RP risk score may correspond to at least a 15% risk for RP >5 Sharp units or at least a 27% risk for RP >3 Sharp units. In an embodiment when the RP risk score range is −1 to 3 a high RP risk score may be an RP risk score greater than about 0.29 which corresponds to at least a 6% risk for RP >5 Sharp units or at least a 16% risk for RP >3 Sharp units. In an embodiment when the RP risk score range is −1 to 3 a high RP risk score may be an RP risk score greater than about 0.78 which corresponds to at least a 10% risk for RP >5 Sharp units or at least a 22% risk for RP >3 Sharp units. In an embodiment when the RP risk score range is −1 to 3, a high RP risk score may be an RP risk score greater than about 1.18 which corresponds to at least a 15% risk for RP >5 Sharp units or at least a 27% risk for RP >3 Sharp units. An RP risk score may be converted to a percentage likelihood of radiographic progression.

By “radiographic progression” is intended alterations, structural alterations or structural damage, or inflammation in one or more joints as observed or measured by any method of evaluating radiographic progression known in the art. Methods of evaluating radiographic progression are known in the art and include, but are not limited to, ultrasound, MRI, and X-rays. Rapid radiographic progression is defined as a change in total Sharp score (ΔTSS) greater than five units over a year.

In various embodiments a radiographic progression risk score is obtained by the steps of obtaining or having obtained a biological sample from a subject with rheumatoid arthritis, determining the MBDA_{adj} for the subject, determining at least one of the serological status of the subject, a BMI surrogate score for the subject and the subject's use of targeted therapy using an interpretation function. The RP risk score is obtained from the subject's MBDA_{adj} factor, a BMI surrogate score factor, and one or more values from the group comprising the serological value of the sample and the targeted therapy value of the sample. In some embodiments the subjects MBDA_{adj} factor, BMI surrogate score factor, serological value and targeted therapy value are all used to determine the RP risk score using an interpretation function. In an aspect the interpretation function for an RP risk score is A+(MBDA_{adj} factor)+(serological value)−(BMI surrogate score factor)−(targeted therapy value), wherein A is defined below herein. In an aspect the RP risk score is 0.92+(0.024*MBDA_{adj})+(0.93 if seropositive)−(0.06×BMI surrogate score)−(0.61 if using a targeted therapy). In an aspect the RP risk score is 0.92+(0.024*MBDA_{adj})+(0.93 if seropositive)+(−0.06×BMI surrogate score)+(−0.61 if using a targeted therapy). In an aspect the RP risk score is 0.92+(0.0241×MBDA_{adj})+(0.928 if seropositive)−(0.0632×BMI surrogate score)−(0.608 if using a targeted therapy). By “A” is intended a value selected from the range of 0.7 to 1.2 and preferably from 0.82 to 1.0, including but not limited to 0.92.

MBDA_{adj} factor=MBDA_{adj} multiplied by a predetermined value. In some aspects the predetermined value may be selected from the group of values ranging from 0.01 to 0.03.

The serological value=a value assigned to subjects based on the serological status of the subject. The serological value for a seropositive subject may range from 0.46 to 1.5. Examples of a serological value may include, but are not limited to, 0.928 and 0.93. A value assigned to seronegative subjects may be 0. A seropositive subject is positive for one or both of rheumatoid factor (RF) and anti-CCP antibodies. (The anti-CCP antibody test can also be represented as the ACPA test.) A seronegative subject has test results for both RF and anti-CCP antibodies that are below the respective threshold limit, when both results are known. It is recognized that a positive score for either RF or anti-CCP antibodies is a score above a pre-determined threshold as low levels of either RF or anti-CCP antibodies may occur in healthy subjects. “Anti-CCP antibodies” include but are not limited to anti-CCP-1 antibodies and anti-CCP-2 antibodies. CCP and cyclic citrullinated peptide are used interchangeably.

The use of BMI data in predicting RP risk has been problematic as BMI is not routinely collected in rheumatology practices, is susceptible to mis-reporting, and may not be as reliable as leptin. Surprisingly we have created a BMI surrogate score factor that is a useful in predicting RP risk. The BMI surrogate score factor=BMI surrogate score multiplied by a predetermined value. In some aspects the predetermined value is in the range of 0.1 to 0.02.

The targeted therapy value is a value assigned to subjects based on the subject's use of targeted therapy. In some aspects the predetermined value assigned to a subject using targeted therapy may be in the range of 1.0 to 0.3; examples of a targeted therapy value may include, but are not limited to, 0.608 and 0.61. A value assigned to subjects not using targeted therapy may be 0. By “targeted therapy” is intended a biologic therapy or a targeted synthetic therapy, including but not limited to a targeted synthetic DMARD.

By “subject” is generally intended a mammal. The term “mammal” includes but is not limited to a human, non-human primate, dog, cat, mouse, rat, cow, horse, pig, sheep, and camel. Mammals other than humans can be advantageously used as subjects that represent animal models of inflammation. A subject may be male, female, adult, immature or young. A subject may be one who has been previously diagnosed or identified as having an inflammatory disease. A subject can be one who has already undergone or is undergoing a therapeutic intervention for an inflammatory disease. A subject may also be one who has not been previously diagnosed as having an inflammatory disease; for example a subject may be one who exhibits one or more symptoms or risks factors for an inflammatory condition, or a subject who does not exhibit symptoms or risk factors for an inflammatory condition, or a subject who is asymptomatic for inflammatory disease.

By a “therapeutic regimen”, “therapy” or “treatment(s)” is intended all clinical management of a subject and interventions, whether biological, chemical, physical or a combination thereof, intended to sustain, ameliorate, improve or otherwise alter the condition of a subject. These terms may be used synonymously. Treatments include, but are not limited to, administration of prophylactics or therapeutic compounds including but not limited to conventional DMARDs, biologic, DMARDs, non-steroidal anti-inflammatory drugs (NSAIDs) such as COX-2 selective inhibitors and corticosteroids), exercise regimens, physical therapy, dietary modification and/or supplementation, bariatric surgery, administration of pharmaceuticals and/or anti-inflammatories (prescription or over the counter), steroids, opiates, cannabinoids, devices or processes intended to act on the vagus and/or other nerves, and any other treatments known in the art as efficacious in preventing, delaying the onset of, or ameliorating disease or a disease effect. A “response to treatment” includes a subject's response to any treatment whether biological, chemical, physical or a combination thereof. A “treatment course” relates to the dosage, duration, extent, etc. of a particular treatment or therapeutic regimen. An initial therapeutic regimen as used herein is the first line of treatment.

In some embodiments, the RP risk score, derived as described herein can be used to predict the risk of radiographic progression in at least one joint as, for example, high, moderate or low. The subject's RP risk score may be compared to a set of RP risk scores in a reference population to determine high, moderate or low risk. The cutoffs used to define the category of RP can vary. The risk score can also change based on the range of the score. For example, a score below zero can represent a low level of risk when a range of −1 to 2 is utilized. Differences can be determined based on the range of score possibilities. For example, if using a score range of −1 to 2, a small score difference can be about 0.01 to about 0.09. If using a score range of 0 to 100, a small score difference can be about 0.5 to about 10. In an embodiment the RP risk score may have a score range from −1 to 3.

In some embodiments, it is not required that the RP risk score be compared to any pre-determined “reference”, “normal”, “control”, “standard”, “healthy”, “pre-disease” or other like index in order for the RP score to provide a quantitative measure of risk in the subject. In some embodiments the RP risk score may be compared to an RP risk score from a different time point. In other embodiments, the RP risk score is compared to a “normal” or “control” level or value, utilizing techniques such as reference or discrimination limits or risk defining thresholds in order to define cut-off points and/or abnormal values for radiographic progression risk. The normal level then is the level found in one or more subjects who are not suffering from the inflammatory disease under evaluation or one or more previously tested subjects who did not exhibit radiographic progression within one year. In some embodiments, the reference value can be derived from one or more subjects who have been exposed to treatment for disease, or from one or more subjects who are at low risk or from subjects who have shown improvements as a result of exposure to treatment.

In some embodiments one or more biomarkers is used to obtain the RP risk score. The RP risk score can provide prognosis and monitoring of disease state and/or disease activity in inflammatory disease and in autoimmune disease. In some embodiments the RP risk score can be used to provide a prognosis or monitoring of disease state and/or disease activity of RA or early RA in response to therapy. In some embodiments the RP risk score can be used to recommend discontinuation of a therapeutic regimen, to recommend no change in a therapeutic regimen, to recommend a new therapeutic regimen or a combination of recommendations.

Identifying the risk of radiographic progression in a subject allows for a prognosis of the disease and thus for the informed selection of, initiation of, adjustment of or increasing or decreasing various therapeutic regimens in order to delay, reduce or prevent that subject's progression to a more advanced disease state. Subjects may be identified as having a particular risk of RP and so can be selected to begin or accelerate treatment to prevent or delay the further progression of inflammatory disease. Subjects may be identified as having a low or moderate risk of RP, and so can be selected to have their treatment decreased or discontinued. In other embodiments subjects may be identified by their RP risk scores as being at a particular risk for RP and can have therapy selected based on RP risk. In other embodiments the direction of change between risk scores obtained at different time points may impact treatment decisions. For example, a subject with a moderate risk of RP at a second time point after a high risk of RP at a first time point may continue with a treatment regimen or the treatment regimen may be decreased. In another example a subject with a moderate risk of RP at a second time point after a low risk of RP at a first time point may administer a different RA therapy.

In regard to the need for early and accurate evaluation of RP risk, recent advances in RA treatment provide a means for more effective disease management and treatment of RA within the first months of symptom onset, often with significantly improved outcomes. However radiographic progression can occur while the subject is experiencing few or minimal RA-related symptoms. The radiographic progression results in damaged joints and may later lead to more severe disability.

### Measurement of Biomarkers

The quantity of one or more biomarkers of the present teachings can be indicated as a value. The value can be one or more numerical values resulting from the evaluation of the sample and can be derived by any method of evaluating the biomarker of interest known in the art. The actual measurement of levels of a biomarker of interest can be determined at the protein or nucleic acid level using any method known in the art. “Protein” detection comprises detection of full-length proteins, mature proteins, pre-proteins, polypeptides, isoforms, mutations, variants, post-translationally modified proteins and variants thereof and can be detected in any method known in the art. Levels of biomarkers may be determined at the protein level directly or indirectly. Methods known in the art include, but are not limited to immunoassays, protease assays, kinase assays, phosphatase assays and expression assays. Biomarkers may be detected at the nucleic acid level also. Such methods include, but are not limited to, RT-PCR, qPCR, amplification-based detection and quantitation methods, ribonuclease protection assays, Northern or Southern blot analysis, quantitatively amplifying biomarker nucleic acid sequences. Alternatively, biomarker protein or nucleic acid metabolites can be evaluated. The term “metabolite” includes any chemical or biochemical product of a metabolic process such as any compound produced by the processing, cleavage or consumption of a biological molecule. Methods of evaluating metabolites are known in the art and include, but are not limited to, refractive index spectroscopy, UV-spectroscopy, fluorescence analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, ion spray spectroscopy combined with mass spectrometry, capillary electrophoresis, IR detection, and fluorescent dye based assays.

### Therapeutic Regimens

The present invention provides methods of treating a patient having RA who has a high RP risk score with appropriate RA therapy and methods of treating a patient having RA who has a low or moderate RP risk score by maintaining or reducing said RA therapy. Determining a patient's RP risk score at multiple time points can provide a clinician with a dynamic picture of a subject's biological state. The RP risk score can thus provide subject-specific biological information which will be informative for therapy decisions and will facilitate therapy response monitoring and should result in more rapid and more optimized treatment, better control of disease and increase in the proportion of subjects achieving remission.

Treatment strategies for autoimmune disorders are confounded by the fact that some autoimmune disorders, such as RA, encompass a diverse array of related symptoms that can flare or go into remission. The complexity of the RA physiology may directly relate to no single therapy having proven optimal for treatment. As the number of therapeutic options increases, there is a need for individually tailored treatment. However, some subjects appear to be in remission while radiographic progression is occurring or is likely to occur. The undetected or unnoticed radiographic progression can result in significant joint deterioration that could have been prevented or reduced by more effective therapies. In patients with early RA (eRA), methotrexate (MTX) is usually recommended as a first line treatment and in non-responders the addition of other conventional synthetic (non-biological) DMARD therapies (e.g., to create so-called triple DMARD therapy) or of biological (e.g., anti-TNF) therapy are both supported by data and recommended. Identifying patients with a high risk of radiographic progression would lead to more personalized medicine and increased effectiveness of therapy.

Therapies may be conventional synthetic disease modifying anti-rheumatic drugs (DMARDs), biologic DMARDs, targeted synthetic DMARDs or alternative therapies. Examples of conventional synthetic DMARDs include, but are not limited to, methotrexate (MTX), azathioprine (AZA), bucillamine (BUC), chloroquine (CQ), ciclosporin (CSA, cyclosporine or cyclosporin), doxycycline (DOXY), hydroxychloroquine (HCQ), intramuscular gold (IM gold), leflunomide (LEF), levofloxacin (LEV), sulfasalazine (SSZ), folinic acid, D-pencillamine, gold auranofin, gold aurothioglucose, gold thiomalate, cyclophosphamide and chlorambucil. Examples of biologic therapies may include, but are not limited to, the TNF inhibitors (including but not limited to infliximab, adalimumab, etanercept, golimumab, certolizumab), IL-1 inhibitors (including but not limited to anakinra), T-cell modulators (including but not limited to abatacept), B-cell modulators (including but not limited to rituximab), IL-6 receptor and IL-6 inhibitors (including but not limited to tocilizumab and sarilumab). Examples of targeted synthetic DMARDs may include but are not limited to janus kinase (JAK) inhibitors. JAK inhibitors include, but are not limited to, tofacitinib, baricitinib and upadicitinib.

In many embodiments the RP risk score is compared to a reference standard in order to direct treatment decisions. The reference standard used for any embodiment disclosed herein may comprise average, mean or median levels of RP risk scores from a control population. The reference standard may further include an earlier time point from the same subject. For example, a reference standard may include a first time point, and the RP risk score may be determined again at a second, third, fourth, fifth, sixth, seventh time point or more. Any time point earlier than any particular time point can be considered a reference standard. The reference standard may additionally comprise cutoff values or any other statistical attribute of the control or earlier time points of the same subject, such as a standard deviation from the mean levels of similar patients who did or did not experience radiographic progression. In some embodiments, the control population may comprise healthy individuals or the same subject prior to the administration of a particular therapy.

In some embodiments an RP risk score may be obtained from the reference time point, and a different score may be obtained from a later time point. A first time point can be when an initial therapeutic regimen or a revised therapeutic regimen is begun or when a first immunoassay is performed. Intervals between time points may be days, months, years, etc. In some embodiments an interval between time points is two, three, four, five, six, seven, eight, nine, ten, eleven, twelve, eighteen, twenty-four, thirty, thirty-six months; four, five, six, seven, eight, nine, ten, eleven, twelve, thirteen, fourteen, fifteen or more years.

A difference in the RP score between timepoints can be interpreted as an increase or decrease in risk. For example, a lower score at a second time point may indicate a lower level of risk and a higher score at a second time point may indicate a higher level of risk.

### Reference Therapy for Treatment

In some embodiments, a patient is treated more aggressively or less aggressively than a reference therapy based on the RP risk score. A reference therapy is any therapy that is the standard of care for treatment. The standard of care for RA may vary temporally and geographically.

In some embodiments, a more aggressive therapy than the standard therapy comprises beginning treatment earlier than is the standard for therapy. In some embodiments, a more aggressive therapy than the standard therapy comprises administering more frequent doses, higher doses, more frequent and higher doses, a combination of therapies, at least one additional therapy with a standard therapy, a therapy that is usually used only after certain other types of therapy have been used, or a therapy with more risk of adverse effects than the standard therapy. In various embodiments an aggressive therapy may comprise an altered combination of drugs, an altered drug dosing schedule, or an altered length of therapy.

In some embodiments, a less aggressive therapy than the standard therapy comprises delaying treatment relative to the standard therapy. In some embodiments a less aggressive therapy than the standard therapy comprises administering less frequent doses, lower doses, less frequent and lower doses, selecting less potent medicines, a smaller number of treatments used in combination, a smaller number of compounds in a combination therapy, or substituting a standard therapy with a less potent therapy, a shortened length of therapy, a therapy with lower risk of adverse effects, or no therapy at all. In some embodiments a less aggressive therapy is the standard therapy. RA therapy may involve drug selection, drug combination, drug dose, dosing frequency and length of therapy.

Standard therapies for RA include, but are not limited to, conventional synthetic DMARDs, corticosteroids, biologics, targeted synthetic DMARDS and other immunosuppressive agents. Immunosuppressive agents repress the patient's natural immune response and may increase the risk of infection or illness. The risks associated with long-term use of NSAIDS and other pain-relieving agents are well recognized in the art and include but are not limited to peptic ulcer disease, nephrotoxicity and hepatoxicity. Adverse effects of RA therapies, including but not limited to a side effect or side effects, may vary depending on the specific therapy and dosing regimen. Adverse effects of RA therapies may include, but are not limited to, infection, immunosuppression, lymphoma, cancer, autoimmune disease, hepatoxicity, pulmonary toxicity and kidney disease.

### Systems for Implementing Risk Assessment Tests

Tests for measuring risk according to various embodiments may be implemented on a variety of systems typically used for obtaining test results, such as results from immunological or nucleic acid detection assays. Such systems may comprise modules that automate sample preparation, that automate testing such as measuring biomarker levels, that facilitate testing of multiple samples and/or are programmed to assay the same test or different tests on each sample. In some embodiments the testing system comprises one or more of a sample preparation module, a clinical chemistry module and an immunoassay module on one platform. Testing systems are typically designed such that they also comprise modules to collect, store and track results, such as by connecting to and utilizing a database residing on hardware. Examples of these modules include physical and electronic data storage devices known in the art, such as a hard drive, flash memory, magnetic tape, solid state drive, USB flash media, SD cards, CD, DVD, BluRay discs, and cloud storage. Test systems also generally comprise a module for reporting and/or visualizing results. Some examples of reporting modules include a visual display or graphical user interface, links to a database, a printer, etc.

Embodiments may comprise a system for predicting risk for radiographic progression in a subject. In some embodiments, the system employs a module for applying a formula to an input comprising the measured levels of biomarkers in a panel and outputting a score. In some embodiments, the measured biomarker levels are test results which serve as inputs to a computer that is programmed to apply the formula. The system may comprise other inputs in addition to or in combination with biomarker results in order to derive an output score; e.g. one or more clinical parameters such as therapeutic regimen, tender joint count (TJC), swollen joint count (SJC), morning stiffness, arthritis of a certain number of joint areas (such as but not limited to, three or more joint areas), arthritis of hand joints, symmetric arthritis, rheumatoid nodules, radiographic changes and other imaging, gender/sex, age, race/ethnicity, disease duration, height, weight, body-mass index, family history, CCP status, RF status, ESR, smoker/non-smoker, etc. In some embodiments the system can apply a formula to biomarker level inputs and then output a risk score that can then be analyzed in conjunction with other inputs such as other clinical parameters. In other embodiments, the system is designed to apply a formula to the biomarker and non-biomarker inputs (such as clinical parameters) together and then report a composite output risk index.

A number of testing systems are presently available that may be used to implement various embodiments. These systems include, but are not limited to, the VECTRA™, ARCHITECT™, integrated immunochemistry systems, high-throughput automated clinical chemistry analyzers, VITROS™, chemistry analysis apparatuses used to generate test results from blood and other body fluids, DIMENSION™ and systems for analysis of body fluids comprising computer software and hardware for operating the analyzers.

A kit for determining an RP risk score may comprise one or more biomarker detection reagents packaged together for conducting any method of the present application. Kit components may include but are not limited to oligonucleotides, oligonucleotides specific to a biomarker of interest, MBDA_{adj} test components, fragments of a biomarker nucleic acid, antibodies, including antibodies to a biomarker protein or to a protein encoded by a biomarker nucleic acid, aptamers, separate containers of a nucleic acid or antibody, an antibody attached to a solid matrix, components to attach an antibody to a solid matrix, control formulations, a detectable label, Northern hybridization components, components for sandwich ELISA or other types of immunoassay, and instructions for generating an RP risk score. A detectable label may be selected from the group comprising but not limited to, fluorescein, green fluorescent protein, rhodamine, cyanine dyes, Alexa dyes, luciferase, and radiolabels. A kit may comprise sample collection components including but not limited to a sample collection reagent, a sample collection vessel, and a sample collection container. A kit may comprise a serological status component. Serological status components include, but are not limited to, a RF antibody, an anti-CCP antibody, an ACPA antibody, an antibody to a solid matrix, control formulations, a detectable label, Northern hybridization components, and components for sandwich ELISA or other types of immunoassay.

In some embodiments a biomarker detection reagent may be immobilized on a solid matrix, such as but not limited to, a porous strip. The solid matrix may include a plurality of sites containing a nucleic acid, a site for a positive control, a site for a negative control or control sites may be provided on a separate solid matrix. The detection sites may be configured in any suitable arrangement.

In some embodiments the kit may comprise a nucleic acid substrate array comprising one or more nucleic acid sequences. The nucleic acids on the array may target one or more nucleic acid sequences represented by a biomarker in the MBDA panel. In some embodiments the substrate array can be on a solid substrate such as a chip or on a solution array. See U.S. Pat. No. 5,744,305; xMAP (Luminex), RayBio Antibody Arrays (RayBiotech), CellGard (Vitra Biosciences) and Quantum Dots' Mosaic (Invitrogen).

A machine-readable storage medium may comprise a data storage material that is encoded with machine-readable data or data arrays. The data and machine-readable storage medium are capable of being used for a variety of purposes, when using a machine programmed with instructions for such data. Such purposes may include, but are not limited to, storing, accessing and manipulating information relating to the risk of a subject or population over time or in response to treatment or for drug discovery for inflammatory disease, etc. Data comprising measurements of biomarkers and/or the evaluation of radiographic progression or RP risk scores can be implemented in computer programs that are executing on programmable computers which comprise a process, a data storage system, one or more input devices and one or more output devices. Program code may be applied to the input data to perform the functions described herein and to generate output information. This output information may be applied to one or more output devices. The computer may be a personal computer, a microcomputer or a workstation of convention design.

The computer programs can be implemented in a high-level procedural or object-oriented programming language to communicate with a computer system. The programs may also be implemented in machine or assembly language. The programming language may be a compiled or interpreted language. Each computer program may be stored on storage media or a device such as ROM, magnetic diskette, flash drive or server, and can be readable by a programming computer for configuring and operating the computer when the storage media or device is read by the computer to perform the described procedures. Any health-related data management system may be considered to be implemented as a computer-readable storage medium, configured with a computer program where the storage medium causes a computer to operate in a specific manner to perform various functions as described therein.

A BMI surrogate score may be obtained from leptin, sex, sex², and age; a BMI surrogate score methodology correlates well with BMI. The BMI surrogate score was generated from five cohorts by optimizing non-disease related variables.

The practice of the present teachings may also employ conventional methods of statistical analysis within the skill of the art. Such techniques are explained fully in the literature. See, e.g., J. Little and D. Rubin, Statistical Analysis with Missing Data, 2nd Edition 2002, John Wiley and Sons, Inc., NJ; M. Pepe, The Statistical Evaluation of Medical Tests for Classification and Prediction (Oxford Statistical Science Series) 2003, Oxford University Press, Oxford, UK; X. Zhoue et al., Statistical Methods in Diagnostic Medicine 2002, John Wiley and Sons, Inc., NJ; T. Hastie et. al, The Elements of Statistical Learning: Data Mining, Inference, and Prediction, Second Edition 2009, Springer, NY; W. Cooley and P. Lohnes, Multivariate procedures for the behavioral science 1962, John Wiley and Sons, Inc. NY; E. Jackson, A User's Guide to Principal Components 2003, John Wiley and Sons, Inc., NY.

### EXAMPLES

### Example 1. Development of Multivariate Based Assay

Four RA cohorts were used to develop the multivariate model and assay. Two RA cohorts were used for training (OPERA and BRASS, n=555) and two were used for validation (SWEFOT and Leiden, n=397). Each pair of cohorts was heterogeneous in disease duration and treatment history. BMI data were not available for one validation cohort, so a BMI surrogate was modeled using forward selection with the two training cohorts and 3 others (CERTAIN, InFoRM, RACER) (N=1411 for the five cohorts combined). An RP risk score was then trained using forward selection in a linear mixed-effects regression, considering disease-related and demographic variables as predictors of change in modified total Sharp score over one year (ΔmTSS), with a random effect on cohort. The RP risk score was validated as a predictor of RP with two cutoffs (ΔmTSS >3 and >5) using logistic mixed-effects regression. Odds ratios (OR) and 95% profile likelihood-based confidence intervals (CI) were calculated from the models and significance was assessed by likelihood ratio tests. The OR (CI) for MBDA_{adj} was 0.0241 (0.0101, 0.034). The OR (CI) for seropositivity was 0.928 (0.485, 1.4). The OR (CI) for the BMI surrogate was −0.0632 (−0.114, −0.0133). The OR (CI) for targeted therapy was −0.608 (−1.03, −0.219). Risk curves were generated to show probability of RP as a function of the RP risk score.

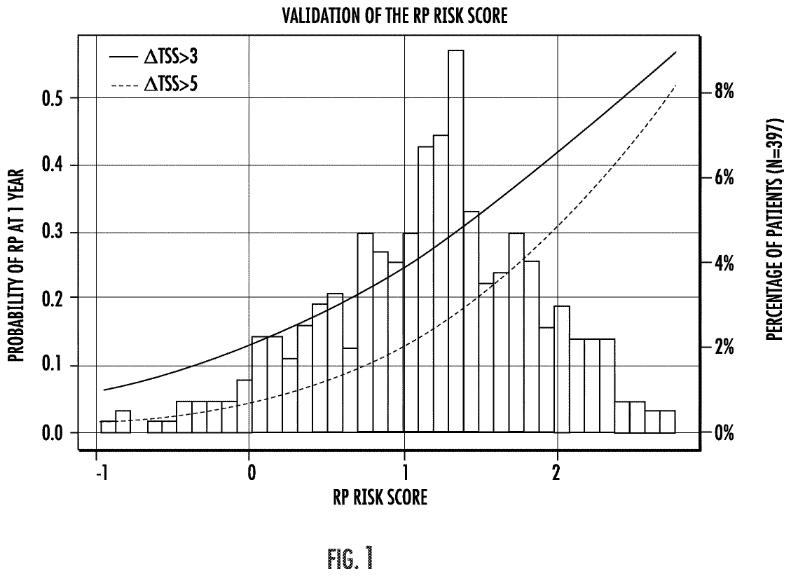

The BMI surrogate score included leptin, sex, age and age² and correlated well with BMI (ρ=0.74). By age² is intended the subject's age in years squared. In training, the most significant independent predictors of RP were MBDA_{adj} (p=0.00020), seropositivity (p=9.3×10⁻⁵), BMI surrogate score (p=0.013) and use of targeted therapy (p=0.0026). The final model was: RP risk score=0.92+(0.0241×MBDA_{adj})+(0.928 if seropositive)−(0.0632×BMI surrogate score)−(0.608 if using a targeted therapy). In validation, the OR (95% CI) of the RP risk score for predicting ΔTSS >3 or >5 were 2.2 (1.6, 3.2) (p=2.6×10⁻⁶) and 3.1 (2.0, 5.1) (p=5.7×10⁻⁸), respectively (FIG. 1). The odds of a patient having RP increases by 50% for each 21-unit or 15-unit increase in MBDA_{adj}, for RP defined as ΔTSS >3 or >5, respectively. See FIG. 1.

The Vanier score is a matrix risk score using the following variables: presence of any erosion, rheumatoid factor (RF) status, categorized swollen joint count (SJC), and categorized C-reactive protein (CRP) levels. See Vanier et al 2019 Rheumatology. Our results indicated the Vanier score was only slightly significant at one RP score (data not shown).

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of obtaining a radiographic progression (RP) risk score for a subject with rheumatoid arthritis (RA), said method comprising: (i) obtaining or having obtained a biological sample from said subject; (ii) determining the multi-biomarker disease activity score adjusted for age, sex and adiposity (MBDAadj) for said subject; (iii) determining at least one of (a) serologic status of said subject, (b) a BMI surrogate score for said subject, and (c) use of targeted therapy; (iv) assigning a serological value if the serological status of said sample is determined; (v) assigning a targeted therapy value, if use of targeted therapy is determined; and obtaining an RP risk score from said subject's MBDAadj factor, a BMI surrogate score factor, and one or more values selected from the group consisting of the serological value of said sample and a targeted therapy value, using an interpretation function.

2. The method of claim 1, wherein a high RP risk score indicates an increased risk of ATSS>.

3. The method of claim 2, wherein a high RP risk score indicates an increased risk of ATSS>.

4. A method for determining if a patient having RA is at increased risk of exhibiting an RP-related effect comprising the steps (i) obtaining or having obtained a biological sample from said subject; (ii) determining the multi-biomarker disease activity score adjusted for age, sex and adiposity (MBDAadj) for said subject; (iii) determining at least one of (a) serologic status of said subject, (b) a BMI surrogate score for said subject, and (c) use of targeted therapy; (iv) assigning a serological value if the serological status of said subject is determined; (v) assigning a targeted therapy value, if use of targeted therapy is determined; and (vi) obtaining an RP risk score from said subject's MBDAadj factor, a BMI surrogate score factor, and one or more values selected from the group consisting of the serological value of said sample and a targeted therapy value using an interpretation function, wherein a high RP risk score indicates said patient is at increased risk of exhibiting an RP-related effect.

5. The method of claim 4, wherein said RP-related effect is selected from the group comprising tissue destruction, cartilage loss, synovial fluid build-up, inflammation and joint erosion.

6. A method for treating a patient with RA with an appropriate RA therapy, the method comprising determining if the patient has a high RP risk score by (i) obtaining or having obtained a biological sample from said patient, (ii) determining the MBDAadj for said patient, (ii) determining the serological status of said patient and assigning a serological value to said subject, (iii) determining a BMI surrogate score factor, (iv) determining if there is use of targeted therapy and assigning a targeted therapy value; obtaining an RP risk score from said MBDAadj, said BMI surrogate score factor and one or more values selected from the group consisting of the serological value of said sample and a targeted therapy value, using an interpretation function; and if the patient has a high RP risk score then administering a different RA therapy to said patient.

7. The method of claim 6, wherein a high RP risk score indicates an increased risk of ATSS>.

8. The method of claim 7, wherein a high RP risk score indicates an increased risk of ATSS>.

9. The method of claim 6, wherein said different RA therapy is selected from the group comprising administering an additional compound, changing a compound, changing the dosing regimen of one or more compounds, or both changing compounds and changing dosing regimen of one or more compounds.

10. The method of claim 6, wherein the risk of an adverse effect for a patient who does not have a high risk of RP is reduced when said RA therapy is reduced and wherein said adverse effect is selected from the group comprising increased risk of infection and hepatoxicity.

11. The method of claim 6, wherein said patient is at increased risk of exhibiting an RP-related effect.

12. The method of claim 6, wherein the risk of exhibiting an RP-related effect is lower when said RA therapy is administered to said patient than it would be if said patient does not receive said RA therapy.

13. The method of claim 11, wherein said RP-related effect is selected from the group comprising tissue destruction, cartilage loss, inflammation and joint erosion.

14. A method of treating a subject with RA, said method comprising determining if said subject is at increased risk of exhibiting an RP-related effect comprising the steps (i) obtaining or having obtained a biological sample from said subject; (ii) determining the multi-biomarker disease activity score adjusted for age, sex and adiposity (MBDAadj) for said subject; (iii) determining at least one of (a) serologic status of said subject, (b) a BMI surrogate score for said subject, and (c) use of targeted therapy; (iv) assigning a serological value if the serological status of said subject is determined; (v) assigning a targeted therapy value, if use of targeted therapy is determined; and (vi) obtaining an RP risk score from said subject's MBDAadj factor, a BMI surrogate score factor, and one or more values selected from the group consisting of the serological value of said sample and a targeted therapy value, using an interpretation function; and if said subject at increased risk of exhibiting an RP-related effect, then administering a different RA therapy.

15. The method of claim 14, wherein said RP-related effect is selected from the group comprising tissue destruction, cartilage loss, inflammation and joint erosion.

16. A method of monitoring treatment efficacy in a subject with RA, said method comprising the steps of (i) obtaining or having obtained a first biological sample from said patient at a first time point; (ii) determining the multi-biomarker disease activity score adjusted for age, sex and adiposity (MBDAadj) for said patient; (iii) determining at least one of (a) serologic status of said patient, (b) a BMI surrogate score for said patient, and (c) use of targeted therapy; (iv) assigning a serological value if the serological status of said subject is determined; (v) assigning a targeted therapy value, if use of targeted therapy is determined; (vi) obtaining a first RP risk score from said subject's MBDAadj factor, a BMI surrogate score factor, and one or more values selected from the group consisting of the serological value of said sample and a targeted therapy value, using an interpretation function; (vii) administering one or more treatment regimens; (viii) obtaining or having obtained a second biological sample from said patient at a second time point; (ix) determining the multi-biomarker disease activity score adjusted for age, sex and adiposity (MBDAadj) for said patient; (x) determining at least one of (a) serologic status of said patient, (b) a BMI surrogate score for said patient, and (c) use of targeted therapy; (xi) assigning a serological value if the serological status of said subject is determined; (xii) assigning a targeted therapy value, if use of targeted therapy is determined; (xiii) obtaining a second RP risk score from said subject's MBDAadj factor, a BMI surrogate score factor, and one or more values selected from the group consisting of the serological value of said sample and a targeted therapy value, using an interpretation function; and (xiv) comparing said first and second RP risk scores to determine treatment efficacy.

17. The method of claim 16, wherein the change between said first RP risk score and said second RP risk score indicates treatment efficacy.

18. A method according to claim 1, wherein said interpretation function is A+(MBDAadj factor)+(serological value)−(BMI surrogate score factor)−(targeted therapy value), wherein A is a value selected from the range of 0.7 to 1.2.

19. A kit for determining an RP risk score for a subject with RA, said kit comprising a sample collection component, a MBDAadj component and a serological status component.
